(19) United States
(12) Patent Application Publication
(10) Pub. No.: US 2022/0133688 A1
(43) Pub. Date: May 5, 2022

(54) MACROCYCLIC LACTONE ANTHELMINTICS AGAINST NEMATODES

(71) Applicant: Bayer Animal Health GmbH, Leverkusen (DE)

(72) Inventors: Daniel KULKE, Leverkusen (DE); Adrian WOLSTENHOLME, Athens, GA (US); Barbara REAVES, Athens, GA (US)

(21) Appl. No.: 17/434,262

(22) PCT Filed: Feb. 28, 2020
(86) PCT No.: PCT/US2020/020491

§ 371 Date: Aug. 26, 2021

Related U.S. Application Data
(60) Provisional application No. 62/811,955, filed on Feb. 28, 2019.

(51) Int. Cl. A61K 31/365; A61K 31/7048; A61P 33/10

(52) U.S. Cl. CPC A61K 31/365; A61P 33/10; A61K 31/7048

(57) ABSTRACT

The present invention relates to a milbemycin for use in control, treatment and/or prevention of infections with nematodes, preferably filariae, more preferably Dirofilaria immitis which are resistant to at least one other macrocyclic lactone anthelmintic. The present invention further relates to the use of milbemycins for stimulating attachment of polymorphonuclear neutrophils (PMNs) and/or peripheral blood mononuclear cells (PBMCs) to larvae of nematodes, as well as to a method for stimulating attachment of PMNs and/or PBMCs to larvae of nematodes. In a further aspect, the present invention relates to an in vitro assay for determining and/or characterizing an agent for control, treatment and/or prevention of an infection with nematodes.

This application claims priority to and the benefit of U.S. Provisional Application No. 62/811,955, filed on Feb. 28, 2019, the entire disclosure of which is incorporated by reference herein in its entirety.

The present invention relates to a milbemycin for use in control, treatment and/or prevention of infections with nematodes, particularly filariae, even more particularly larvae of filariae, wherein said nematodes are resistant to at least one other macrocyclic lactone anthelmintic.

The present invention further relates to the use of milbemycin for stimulating attachment of polymorphonuclear neutrophils (PMNs) and/or peripheral blood mononuclear cells (PBMCs) to nematodes, as well as to a method for stimulating attachment of PMNs and/or PBMCs to nematodes.

In a further aspect, the present invention relates to an in vitro assay for determining and/or characterizing an agent for control, treatment and/or prevention of an infection with nematodes.

Macrocyclic lactone anthelmintics, including avermectins and milbemycins, are an important drug class currently available for the control, treatment and/or prevention of infections with nematodes in a variety of hosts including humans, dogs, cats, cattle, sheep, pigs, and horses. As an example, for the prevention of heartworm disease caused by Dirofilaria immitis, in cats and dogs the macrocyclic lactone anthelmintics are the only registered option. These anthelmintic drugs are highly effective at preventing the development of third stage larvae (L3) from susceptible parasites to adulthood when used at very low doses. At higher doses, they are also effective at removing microfilariae (Mf) from the circulation of infected animals.

Even though anthelmintic administration is well established in fighting parasites, the specific mechanism of action of macrocyclic lactone anthelmintics is still under research especially in filariae. In vitro incubation of larvae, particularly fully susceptible D. immitis larvae, with concentrations of macrocyclic lactone anthelmintics up to 7.000-fold higher than those concentrations present in blood of infected animals have little effect on their motility or ability to migrate. Thus, the extremely high potency of macrocyclic lactone anthelmintics to prevent heartworm disease in vivo is not mirrored by their activity against those larvae in vitro. This has led to speculation that host factors may be required for their full anthelmintic activity in vivo and attention has focused on the immune system.

It was found that rat neutrophil granulocytes isolated after intraperitoneal casein injection of the donors exhibit larvicidal effects in vitro against microfilariae of Litomosoides carinii in the presence of ivermectin, suggesting a participation of host factors in infections with filariae (Zahner et al., 1997). However, reliable in vitro assays capable of reflecting anthelmintic treatment conditions in vivo are still missing.

In recent years, it has been confirmed that D. immitis parasites resistant to at least one anthelmintic of the macrocyclic lactones are circulating in the United States. A growing threat is arising from parasites resistant to standard macrocyclic lactone anthelmintic treatment, such as preventive administration of ivermectin. In vitro assays differentiating resistant from susceptible parasites such as motility and migration assays have proved to be difficult, either at the Mf or L3 (Maclean et al., 2017). On the molecular level, Mf populations resistant towards macrocyclic lactones are suspected to be characterized by very high frequencies of single-nucleotide polymorphisms in a Mf gene encoding a P-glycoprotein transporter, comprised of homozygous guanosine residues at two locations (“GG-GG” genotype) (Geary et al., 2011). Until now, only in vivo Mf suppression tests seem to provide a reliable way of identifying drug-resistant strains without requiring the euthanasia of infected animals.

It was an object of the present invention to provide improved ways of control, treatment and/or prevention of infections with nematodes, e.g. facing the issue of recently occurring drug resistances to anthelmintics. A further object of the present invention was the provision of reliable in vitro assays reflecting anthelmintic treatment conditions in vivo e.g. for differentiating resistant from susceptible parasites and characterizing anthelmintics with respect to their ability to control, treat and/or prevent infections with nematodes.

It was found that milbemycins are suitable agents to overcome drug resistance of nematodes to other macrocyclic lactone anthelmintics, such as avermectins. In this context, it was found that milbemycins stimulate attachment of PMNs and/or PBMCs to nematodes and larvae thereof, thus promoting a cellular immune response which seems to be responsible for inactivating nematodes. These findings can be used in effective in vitro assays for determining or characterizing an agent for control, treatment and/or prevention of an infection with nematodes in vivo.

Accordingly, the present invention provides a milbemycin for use in control, treatment and/or prevention, in particular prevention, of infections with nematodes, which are resistant to at least one other macrocyclic lactone anthelmintic.

Among infections with nematodes, preferably infections with filariae, more preferably infections with larvae of filariae are targeted. Preferred filariae are selected from Dirofilaria immitis, Brugia malayi, Wuchereria bancrofti, Loa loa, Mansonella spp., Dirofilaria repens and/or Onchocerca volvulus, even more preferred Dirofilaria immitis.

The milbemycin may be selected from milbemectin, milbemycinoxim, moxidectin, nemadectin, milbemycin-D and combinations thereof, in particular moxidectin.

The at least one other macrocyclic lactone anthelmintic to which the nematodes are resistant may be selected from avermectins, such as ivermectin, selamectin, doramectin, abamectin, and combinations thereof. In one embodiment, the infecting nematodes are resistant to at least one other macrocyclic lactone, which is ivermectin. In some embodiments said other macrocyclic lactone anthelmintic is selected from milbemycines, such as milbemectin, milbemycinoxim, moxidectin, nemadectin, milbemycin-D, and a combination thereof.

Nematodes which are resistant to other macrocyclic lactone anthelmintics are defined as showing a reduction in effectiveness of a medication with said other macrocyclic lactone anthelmintic.

Such resistance may be due to strong selection events. It has been found that resistant nematodes may be characterized by very high frequencies of single-nucleotide polymorphisms. For example in D. immitis a gene encoding for a P-glycoprotein transporter, comprised of homozygous guanosine residues at two locations (“GG-GG” genotype) (Geary et al., 2011). Thus, in one aspect, nematodes which are resistant to at least one other macrocyclic lactone anthelmintic, such as ivermectin, may have an increased frequency of the GG-GG genotype as described in Bourguinat, 2011 (herein incorporated by reference). Particularly, nematodes, more particularly nematode populations, such as D. immitis, which are resistant to at least one other macrocyclic lactone anthelmintic have a frequency of the GG-GG genotype which is increased as compared to the respective wildtype.

Preferably, nematodes resistant to other macrocyclic lactone anthelmintics are identified in that they establish a patent infection in subjects being infected therewith despite treatment and/or prevention with said other macrocyclic lactone anthelmintics, in particular despite prevention measures according to the label of a commercial product comprising said other macrocyclic lactone anthelmintics. For example, said prevention measure may be a treatment with said other macrocyclic lactone anthelmintics, e.g. for at least 1 month, preferably for at least 6 months.

A nematode resistant to other macrocyclic lactone anthelmintics can also be identified via the determination of an EC₅₀ value of said macrocyclic lactone which is increased by at least 15%, preferably at least 20%, as compared to the EC₅₀ value of said macrocyclic lactone in the respective wild-type nematode. The EC₅₀ value can be determined as known in the art or as described herein. Preferably the EC₅₀ value can be determined using an in vitro assay. For example, the EC₅₀ value can be determined using motility as read-out. Preferably, the EC₅₀ value can be determined using cell attachment as a read-out.

According to the present invention, an infection is defined as patent when direct evidence of nematodes can be detected in the subject's body fluids or discharges, e.g. in the subject's faeces, blood or secretions, regardless of whether symptoms have appeared. Suitable assays for detecting evidence of nematodes are known in the art and e.g. include detection of antibodies against antigens of the infecting nematodes in the subject's blood such as by means of ELISA. Treatment regimens are known to the person skilled in the art and comprise administration of an anthelmintic e.g. once a month, once every 6 months, or once every 12 months by a suitable route of administration such as topically, orally, intravenously or the like in a suitable dose, such as 1-200 μg/kg. Respective tests are e.g. described by Geary, 2011 (herein incorporated by reference). The skilled person is well aware that doses may vary depending on the route of administration, the dosage form, etc. For example, the dose may be higher for sustained release formulations as compared to a burst formulation.

Exemplary nematodes, which are resistant to other macrocyclic lactone anthelmintics, such as avermectins, in particular ivermectin, are e.g. D. immitis strains Yazoo-2013, Metairie-2014 and JYD-34 described by Maclean et al., 2017 and Blagburn et al., 2016 (both herein incorporated by reference).

The milbemycin may be administered to the subject in need thereof in a dose adjusted to give a plasma concentration in the subject of 0.1-100 nM, preferably 0.1-10 nM, more preferably 0.5-3 nM of milbemycin. The amount of milbemycin administered to the subject in need thereof is e.g. dependent on the body weight of the subject to be treated, the frequency of administration, the route of administration, the intended type of use differing e.g. between control, treatment and prevention of infections with nematodes, the stage of infection such as the occurrence of larvae, or the like. A suitable dose might be 1-200 μg/kg, preferably 3-120 μg/kg, more preferably 6-120 μg/kg body weight of the subject to be treated.

The milbemycin is administered to the subject in need thereof in predetermined time intervals or in time intervals dependent on the course of disease, which might e.g. be determined dependent on disease related parameters such as the amount of antibodies present in the subject's blood. Suitable administration intervals may be every month, preferably every 6 months, more preferably every 12 months, e.g. depending on the route of administration and type of formulation.

The milbemycin may be administered orally, topically, or parenterally such as cutaneously, subcutaneously, intramuscularly or intravenously, preferably subcutaneously, topically, or orally. The milbemycin is present in a dosage form suitable for the intended route of administration, e.g. in the form of a solution, suspension, or powder for injection, in the form of a solution, suspension, ointment, cream, or gel for topical administration, or in the form of a solution, suspension, tablet, preferably a highly palatable tablet, or a chewable, e.g. a soft-chew, for oral administration.

In a further aspect, the present invention relates to a pharmaceutical preparation comprising at least one milbemycin, and at least one other macrocyclic lactone anthelmintic, preferably an avermectin. In one embodiment, the pharmaceutical preparation of the invention comprises at least one milbemycin which is moxidectin and at least one other macrocyclic lactone anthelmintic, preferably an avermectin. In one embodiment, the pharmaceutical preparation of the invention comprises at least one milbemycin which is moxidectin and at least one avermectin which is ivermectin. In one embodiment, the pharmaceutical preparation is for use in control, treatment and/or prevention of infections with nematodes, which are in particular resistant to at least one of the macrocyclic lactone anthelmintics used but preferably not to all macrocyclic lactone anthelmintics used. Such combination therapy results in an increased efficacy in subjects to be treated having a plurality of different nematodes that may also include resistant nematodes as described above.

In a still further aspect, the present invention relates to a pharmaceutical preparation comprising at least one milbemycin, preferably moxidectin, and peripheral blood mononuclear cells and/or polymorphonuclear neutrophils. In one embodiment, the pharmaceutical preparation may be for use in control, treatment and/or prevention of infections with nematodes, which are in particular resistant to at least one other macrocyclic lactone anthelmintics. Preferably, the pharmaceutical preparation is for use in prevention of infections with nematodes, which are in particular resistant to at least one other macrocyclic lactone anthelmintics. Such combination therapy assures an effective and prompt attachment of cells and/or neutrophils directly after administration of the pharmaceutical preparation, thereby inactivating the nematodes in a more rapid manner as compared to administration of the at least one milbemycin alone.

The infections with nematodes to be prevented, treated, and/or controlled are the same as described above. Nematodes resistant to at least one macrocyclic lactone anthelmintics are defined as set forth above.

Peripheral blood mononuclear cells and/or polymorphonuclear neutrophils may be derived from the subject to be treated. In another embodiment, the PBMCs and/or PMNs may be derived from another subject, preferably of the same species, which is in particular uninfected. In a preferred embodiment, the cells present in the pharmaceutical preparation are derived from an uninfected subject of the same species. The total amount of PBMCs in the pharmaceutical preparation preferably ranges from 1.000 to 100.000 cells/ml, more preferably from 10.000 to 50.000 cells/ml. The total amount of PMNs in the pharmaceutical preparation preferably ranges from 1.000 to 100.000 cells/ml, more preferably from 10.000 to 50.000 cells/ml. The total amount of cells in the pharmaceutical preparation, including PBMCs and PMNs, preferably ranges from 2.000 to 200.000 cells/ml, more preferably from 10.000 to 100.000 cells/ml.

The milbemycin may be selected from milbemectin, milbemycinoxim, moxidectin, nemadectin, milbemycin-D, and combinations thereof, in particular moxidectin. The at least one other macrocyclic lactone anthelmintic may be selected from avermectins, such as ivermectin, selamectin, doramectin, and abamectin, preferably ivermectin.

The pharmaceutical preparation of the invention might be in the form of a solution such as a solution suitable for injection, a suspension, a paste, an ointment, a chewable, a tablet, preferably a palatable tablet, or a granulate. The route of administration might be orally, topically, or parenterally such as cutaneously, subcutaneously, intramuscularly or intravenously, preferably intravenously, topically, or orally. The pharmaceutical preparation is administered in an amount comprising a pharmaceutically effective amount of active agent, resulting e.g. in a plasma concentration of 0.1-100 nM, preferably 0.1-10 nM, more preferably 0.5-3 nM of active agent.

The pharmaceutical preparation of the invention may further comprise at least one other insecticide, such as imidacloprid, pyrantel, or salts thereof. The pharmaceutical preparation of the invention may further comprise at least one pharmaceutically acceptable excipient, selected from the group consisting of fillers, binders, thickeners, disintegrants, lubricants, solvents, buffers, isotonic agents, or mixtures thereof. Suitable fillers are e.g. lactose, cellulose, or starch. Suitable thickeners and binders are e.g. xanthan gum, alginate, cellulose derivatives such as carboxymethyl cellulose, or polyvinylpyrrolidone. Suitable disintegrants are e.g. sodium croscarmellose or sodium bicarbonate. Suitable solvents are e.g. aqua ad injectabilia. Suitable buffers are e.g. phosphate buffers or carbonate buffers.

In a further aspect, the present invention refers to a method for stimulating attachment of polymorphonuclear neutrophils and/or peripheral blood mononuclear cells to nematodes, particularly filariae, even more particularly to larvae of filariae comprising: adding a preparation comprising at least one milbemycin, in particular moxidectin, and PBMCs and/or PMNs to a composition comprising nematodes, particularly filariae, even more particularly larvae of filariae.

The preparation comprising at least one milbemycin, and PBMCs and/or PMNs may be the same as described above.

The composition may comprise larvae of trematodes, cestodes, or nematodes, particularly larvae of nematodes, even more particularly larvae of filariae. Preferred nematodes are described above. The composition preferably comprises about 1-1.000 larvae.

Any PBMC and/or PMN which is—according to optical analysis—in direct contact with one motile larvae for a predetermined time of e.g. at least 1, preferably at least 2 minute(s), is regarded as “attached”. “Stimulating attachment” in the sense of the present invention corresponds to the induction of a statistically significant increase in the attachment of PBMCs and/or PMNs to larvae in comparison to a control sample. In the control sample attachment is assessed in the complete absence of anthelmintics or in the absence of anthelmintics to which the assessed nematodes are susceptible. Preferably, an increase of at least 20%, even more preferably at least 30% is assessed. The method may be conducted in vitro.

Further, the present invention refers to the use of a milbemycin, particularly moxidectin, for stimulating attachment of polymorphonuclear neutrophils and/or peripheral blood mononuclear cells to nematodes, particularly filariae, even more particularly larvae of filariae.

Moreover, the present invention refers to a milbemycin, particularly moxidectin, for use in stimulating attachment of polymorphonuclear neutrophils and/or peripheral blood mononuclear cells, to nematodes, particularly filariae, even more particularly larvae of filariae as described above.

The inventors have found that attachment of polymorphonuclear neutrophils and/or peripheral blood mononuclear cells to nematodes, particularly filariae, even more particularly larvae of filariae correlates with the inactivation of nematodes.

Thus, in a further aspect, the present invention refers to an in vitro assay for determining or characterizing an active agent for control, treatment and/or prevention of an infection with nematodes, preferably filariae, even more preferably larvae of filariae, comprising the steps of
(i) providing larvae of an isolated nematode,
(ii) contacting the larvae of step (i) with PBMCs and/or PMNs and an agent to be determined,
(iii) incubating the mixture obtained in (ii) for a predetermined time,
(iv) measuring the percentage of motile larvae having at least one PBMC or PMN attached,
(v) comparing the percentage obtained in step (iv) with the percentage of motile larvae having at least one PBMC or PMN attached in a control sample, and
(vi) selecting the active agent which has an increased percentage over the control sample by at least 10 preferably at least 20%, more preferably at least 25%.

The larvae provided in step (i) may be selected from nematode isolates as described above. Larvae isolation may be performed by known methods, e.g. as described by Franks et al., 1945. Typically, 1-1.000 larvae, more preferably 10-500 larvae are provided in step (i).

In step (ii) the larvae of step (i) are contacted with a composition comprising PBMCs and/or PMNs and the agent. Preferably, the composition further comprises serum. Typically, the composition bears about 10.000-50.000 cells (PBMCs and/or PMNs). Preferably, the agent is present in the composition in a concentration of 0.1-10.000 nM, more preferably 1-2.000 nM. Step ii) may comprise mixing the larvae of step (i) with the composition. The ratio of number of larvae to total number of PBMCs and/or PMNs in the mixture obtained may be 1:100-1:1000, preferably 1:100 to 1:300. The PBMCs and/or PMNs are preferably derived from the same species, even more preferably from an uninfected subject of the same species. The agent is preferably suspected to act as an anthelmintic.

Incubating the mixture obtained in step (ii) for a predetermined time in step (iii) may take place at standard cell culture conditions such as e.g. 37° C., 5% CO₂, and/or 95% humidity in a cell culture incubator. The predetermined incubation time may be up to 7 days, preferably up to 4 day, more preferably 6-48 h.

In step (iv) the percentage of motile larvae having at least one PBMC or PMN attached after step (iii) is measured. Such measurement may be performed by visual determination, e.g. microscopically or by video analysis, of the number of motile larvae having at least one PBMC and/or PMN attached and the total number of motile larvae in a sample.

For the control sample, steps (i)-(iv) are repeated in the identical manner as described above, except that the composition according to step (ii) is free of any agent to be determined.

In step (v) the percentage obtained in step (iv) is compared with the percentage of motile larvae having at least one PBMC or PMN attached obtained for the control sample.

In step (vi) an agent might be selected as active agent if the percentage obtained in step (iv) is increased over the percentage of the control by at least 10%, preferably at least 20%, more preferably at least 25%.

Surprisingly, it has been found that such selected active agent typically shows also activity against filariae in vivo. Thus, attachment of PMNs and/or PBMCs to larvae plays a relevant role in the mechanism of preventing patent infections with filariae in vivo. The in vitro assay of the present invention, thus, can be used as a reliable indicator in selecting active agents which show in vivo activity against infections with nematodes, particularly filariae, even more particularly larvae of filariae, particularly D. immitis. The assay of the invention is particularly suitable for predicting efficacy of agents against isolates of D. immitis.

Steps (i)-(iv) may be performed several times with varying concentrations of the same agent to be determined. The percentage of motile larvae having at least one PBMC and/or PMN attached may be determined as a function of the concentration of the agent. Such experiments allow the skilled person to determine the EC₅₀ value of the agent. The EC₅₀ value refers to the concentration of an agent which induces a response halfway between the baseline and maximum (defined as 100% effect) after a specified exposure time. It is commonly used as a measure of the agent's activity. EC₅₀ is expressed as a concentration in nmol/l.

Thus, in a further aspect, the present invention refers to an in vitro assay for characterizing an agent for control, treatment and/or prevention of an infection with nematodes, preferably filariae, even more preferably larvae of filariae comprising the steps of
(i) providing larvae of an isolated nematode,
(ii) contacting the larvae of step (i) with PBMCs and/or PMNs and the agent to be determined,
(iii) incubating the mixture obtained in (ii) for a predetermined time,
(iv) measuring the percentage of motile larvae having at least one PBMC or PMN attached at predetermined concentrations of the agent, and
(v) determining an EC₅₀ value of the active agent.

Steps (i)-(iv) may be performed as described above. Determining an EC₅₀ value of the active agent in step (v) is based on the percentages obtained in step (iv) for multiple assay cycles (i)-(iv) performed with varying concentrations of the active agent, such as e.g. multiple equally distributed concentrations in the rage of 0-20 μM. Determination of the EC₅₀ value may be performed by conventional mathematical tools as known in the art.

The EC₅₀ value may be used for comparing different potential agents (candidates) e.g. in view of their anthelmintic characteristics. An increased EC₅₀ value means less activity against nematodes and vice versa.

In a further aspect, the present invention refers to a reagent kit comprising
a) a composition comprising PBMCs and/or PMNs, and
b) a composition comprising larvae of nematodes, preferably filariae, even more preferably larvae of filariae.

The composition b) may be the same as the mixture obtained after step (i) as described above. Composition a) comprises PBMCs and/or PMNs as described above and optionally serum as described above.

The invention is further elucidated by the following examples and figures.

### FIGURES

FIG. 1: Effect of ivermectin and moxidectin on PMN and PBMC attachment to larvae of the D. immitis isolates Missouri (MO) and Georgia-2 (GA-2). A) Effect of ivermectin on the attachment of PMNs. B) Effect of moxidectin on the attachment of PMNs. C) Effect of ivermectin on the attachment of PBMCs. D) Effect of moxidectin on the attachment of PBMCs. For each panel the bars represent the percentage of Mf with at least one cell attached at varying concentrations of the drug; from left to right these were 0, 1, 3, 10, 30, 100, 300 and 1000 nM. MO=Missouri, GA-2=Georgia-2.

FIG. 2: Effect of ivermectin and moxidectin on PMN and PBMC attachment to larvae of D. immitis isolates with suspected resistance against at least one macrocyclic lactone. A) Effect of ivermectin on the attachment of PMNs. B) Effect of moxidectin on the attachment of PMNs. C) Effect of ivermectin on the attachment of PBMCs. D) Effect of moxidectin on the attachment of PBMCs. For each panel the bars represent the percentage of Mf with at least one cell attached at varying concentrations of the drug; from left to right these were 0, 1, 3, 10, 30, 100, 300 and 1000 nM. YAZ=Yazoo-2013, MET=Metairie-2014.

### EXAMPLES

### Methods

### 1. Parasites

The Missouri isolate of D. immitis was provided by the NIH/NIAID Filariasis Research Reagent Resource Center. The Georgia-2 isolate was provided by TRS Labs Inc., Athens, Ga. The Yazoo-2013 and Metairie-2014 strains have been described previously (Maclean et al., 2017).

For microfilariae isolation blood from infected dogs was received in heparinized tubes and centrifuged for 30 minutes at 1200×g at room temperature. The top layer of plasma was removed, and the mass of red blood cells and Mf was brought back to its original volume with 4:1 3.8% (w/v) saline-citrate (38 mg sodium citrate/100 mL physiological saline). 15% (w/v) (1.5 g) saponin was mixed with deionized water (10 mL) and was added (1 mL) for every 15 mL of original volume and the tube was shaken for 30 seconds. The mixture was centrifuged for 30 minutes at 1200×g. The supernatant was discarded, and sodium-citrate was used to bring back to the original volume (15 mL). The mixture was then centrifuged for 4 minutes at 1200×g. The worm mixture was transferred to a new conical tube and mixed with 1× PBS (10 mL). The PBS and Mf mixture was centrifuged for 5 minutes at 1200×g to pellet the Mf and the supernatant was removed. The pellet was resuspended in Roswell Park Memorial Institute (RPMI) cell culture medium prior to assessment of worm numbers.

### 2. Cell Isolation

Blood from an uninfected dog was drawn from jugular punctures and put into heparinized tubes. Blood (10 mL) was transferred from the heparinized tube into a sterile, endotoxin free conical tube (50 mL) with 1:1 PBS. The blood was then underlaid with Histopaque® 1077 (5-10 mL) with an 18-gauge needle and a sterile syringe. The gradient was then centrifuged at 400×g at room temperature for 25 minutes with the brake off. The top layer of plasma was discarded and the middle layer, the PBMC layer, was placed in a separate sterile conical tube. 40 ml ACK buffer (155 mM ammonium chloride, 10 mM potassium hydrogen carbonate, 0.1 mM EDTA, pH 7.3) was added to the red blood cell/PMN mixture and gently mixed by inverting the tube. The blood mixture was set to rest for 5 minutes at room temperature in order for the red blood cells to lyse. The mixture was centrifuged for 5 minutes at 400×g to pellet PMNs. The PMN pellet was washed with PBS and re-suspended in 10 ml PBS-2% BSA then centrifuged again at 400×g for 5 minutes. After removing the supernatant, the pellet was re-suspended in 500 μl RPMI and 500 μl serum.

### 3. Cell Attachment Assays

Assays were set up in triplicate in a 96-well plate with a minimum of 5 biological replicates for each strain. Each biological replicate is defined as independent Mf isolations in different weeks from the same dog. Each well contained 100 Mf of the strain under test, 20,000 cells (PBMC or PMN) and 10% uninfected dog serum in RPMI. The drug concentrations tested were 1, 3, 10, 30, 100, 300 and 1,000 nM, plus a vehicle (1% DMSO) control. The assays were incubated at 37° C. in a 5% CO₂ atmosphere for 24 h (PMN) or 40 h (PBMC), before being visually scored. Attachment in this assay was defined by a motile Mf having at least one cell attached. Static worms were considered to be dead and were not counted.

### 4. Data Analysis

Data were analyzed using Graphpad Prism®, v5 (GraphPad Software, INC., San Diego, Calif.). Cell attachment within each strain was compared using 2-way ANOVA and Tukey's post-hoc test.

### Results

### Example 1

### PMN and PBMC Attachment to Mf in the Presence of Ivermectin

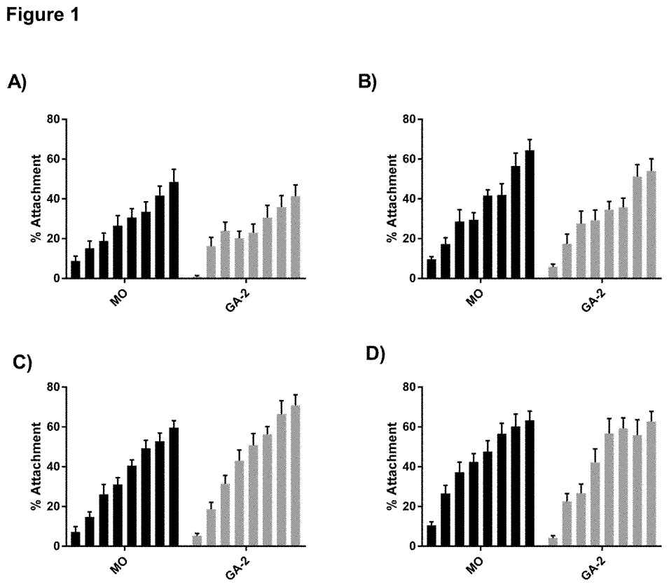

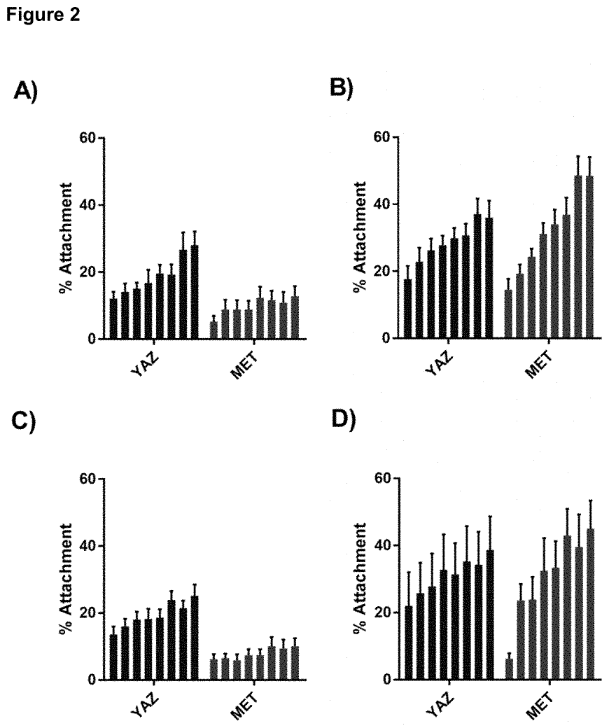

When purified canine PMNs and PBMCs isolated from uninfected dogs are cultured with D. immitis Mf a low percentage of the parasites had cells attached to them after 16 h (PMNs) or 40 h (PBMCs). The addition of ivermectin to the cultures increased the proportion of the Mf with both PMNs and PBMCs attached in a concentration-dependent manner for nearly all the strain/cell type combinations tested, though the concentration at which a statistically significant increase over the no-drug controls was observed did vary between strains (cf. FIGS. 1 and 2).

For the Missouri and Georgia-2 isolates, both of which are susceptible to macrocyclic lactone anthelmintics, 1-3 nM ivermectin was sufficient to cause a significant increase in attachment of both PMNs and PBMCs. For the resistant Metairie-2014 and Yazoo-2013 isolates, higher drug concentrations were required, 100-300 nM for Yazoo-2013 and 1 μM or greater for Metairie-2014 (cf. Table 1); there was no significant increase in attachment of PBMCs to the Metairie-2014 Mf at any concentration of ivermectin. It was found that the maximum percentage of the Mf with cells attached varied between the strains, even at the highest concentration tested (1 μM) (Table 2). 13% of the Metairie-2014 Mf had PMNs attached compared to 48% of the Missouri Mf; for the PBMCs the range was 10% of Metairie-2014 to 71% of the Georgia-2 Mf having cells attached.

### Example 2

### PMN and PBMC Attachment to Mf in the Presence of Moxidectin

The attachment experiments were repeated using moxidectin instead of ivermectin. Likewise, a concentration dependent increase in attachment to the Missouri and Georgia-2 with both PMNs and PBMCs was found (cf. FIGS. 1 and 2). However, a concentration-dependent increased cell attachment to the Metairie-2014 Mf was also observed with moxidectin, in contrast to ivermectin. The concentration of moxidectin at which a significant increase in attachment was observable with this strain was lower than with ivermectin and the maximum level of attachment was higher; both were similar to the values of the susceptible strains. The concentration of moxidectin at which increased attachment was observed for Yazoo-2013 was similar to all the other strains at 3-10 nM (Table 1), but the proportion of the Mf with cells attached tended to be lower (Table 2).

TABLE 1
The lowest drug concentration [ivermectin (IVM) or moxidectin (MOX)]
at which a statistically significant increase (p = <0.05), judged
by 2-way ANOVA, in cell attachment was observed compared to the no-drug control.

| Strain | Missouri | | Georgia-2 | | Yazoo-2013 | | Metairie-2014 | |
|---|---|---|---|---|---|---|---|---|
| Drug | IVM | MOX | IVM | MOX | IVM | MOX | IVM | MOX |
| PMN | 3 nM | 3 nM | 1 nM | 3 nM | 100 nM | 3 nM | 1 μM | 10 nM |
| PBMC | 3 nM | 1 nM | 1 nM | 1 nM | 100 nM | 10 nM | 100 nM | 1 nM |

TABLE 2
Percentage (±SEM) of Mf with cells attached after incubation with 1 μM ivermectin
(IVM) or moxidectin (MOX), rounded to the nearest whole number.

| Strain | Missouri | | Georgia-2 | | Yazoo-2013 | | Metairie-2014 | |
|---|---|---|---|---|---|---|---|---|
| Drug | IVM | MOX | IVM | MOX | IVM | MOX | IVM | MOX |
| PMN | 48 ± 6% | 65 ± 5% | 41 ± 6% | 54 ± 6% | 25 ± 5% | 28 ± 5% | 13 ± 3% | 42 ± 6% |
| PBMC | 60 ± 3% | 63 ± 5% | 71 ± 5% | 63 ± 5% | 18 ± 4% | 19 ± 3% | 10 ± 2% | 49 ± 8% |

If the leukocyte attachment is relevant to the drug's in vivo anthelmintic efficacy, then we would predict that attachment would be reduced in the presence of the drug to Mf of resistant strains as opposed to those of susceptible ones. In general those predictions were supported by the data we obtained. Ivermectin and moxidectin both increased cellular attachment to the Missouri and Georgia-2 Mf at very low concentrations (<10 nM) which correspond to those reported to be present in the plasma of treated dogs. The effects of ivermectin on attachment to the resistant Metairie-2014 and Yazoo-2013 isolates were much less marked (FIG. 2). These results suggest that the in vitro drug-promoted leukocyte attachment is indeed relevant to the full in vivo potential of the macrocyclic lactone anthelmintics and indicates that the host immune response is required for the prevention provided by these drugs. They also suggest that the Mf are a suitable surrogate life-cycle stage for studying resistance in D. immitis, supporting the utility of the present in vitro Mf suppression assay as a diagnostic tool for resistance in infected subjects.

Moxidectin was more effective at promoting attachment to the Metairie-2014 and Yazoo-2013 Mf than was ivermectin (Table 1).

### CITED DOCUMENTS

Blagburn et al., Parasites & Vectors 9, 191, 2016.

Bourguinat et al., Vet. Parasitol 176, 374-381, 2011.

Franks et al., J. Parasitol 31, 158-162, 1945.

Geary et al., Top. Companion Anim. Med. 26, 186-192, 2011.

Maclean et al., Parasites & Vectors 10 (Suppl 2), 480, 2017.

Zahner et al., Experimental Parasitology, 86(2), 110-117, 1997.

1. A method for the control, treatment and/or prevention of infections with nematodes which are resistant to at least one other macrocyclic lactone anthelmintic comprising administering a milbemycin to a subject in need thereof.

2. The method according to claim 1, wherein the milbemycin is selected from the group consisting of milbemectin, milbemycinoxim, moxidectin, nemadectin, milbemycin-D, and combinations thereof.

3. The method according to claim 1, wherein the infections with nematodes are selected from infections with filariae or larvae of filariae.

4. The method according to claim 1, wherein the at least one other macrocyclic lactone anthelmintic is selected from the group consisting of ivermectin, selamectin, doramectin and abamectin.

5. The method according to claim 1, wherein a resistant nematode is defined as exhibiting an EC₅₀ value of the at least one other macrocyclic lactone which is increased by at least 15% as compared to the EC₅₀ value of the wild-type nematode.

6. The method according to claim 1, wherein the subject in need thereof is infected with nematodes which are resistant to at least one other macrocyclic lactone anthelmintic that was previously administered to the subject to treat nematodes.

7. The method according to claim 1, wherein the milbemycin is administered to the subject in need thereof in a dose adjusted to give a plasma concentration in the subject of 0.1-100 nM of milbemycin.

8. The method according to claim 1, wherein the milbemycin is administered to the subject in need thereof every month, every 6 months, or every 12 months.

9. The method according to claim 1, wherein milbemycin is administered orally, topically, or parenterally.

10. (canceled)

11. A pharmaceutical composition comprising at least one milbemycin and a second agent selected from the group consisting of a macrocyclic lactone, peripheral blood mononuclear cells (PBMCs), polymorphonuclear neutrophils (PMNs), and a combination thereof.

12. (canceled)

13. A method for stimulating attachment of polymorphonuclear neutrophils (PMNs) and/or peripheral blood mononuclear cells (PBMCs) to nematodes, or filariae, or larvae thereof, comprising adding a composition comprising at least one milbemycin and PBMCs and/or PMNs to a composition comprising nematodes, or filariae or larvae thereof.

14. (canceled)

15. (canceled)

16. An in vitro assay for determining or characterizing an active agent for control, treatment and/or prevention of an infection with nematodes comprising
(i) providing larvae of an isolated nematode,
(ii) contacting the larvae of (i) with PBMCs and/or PMNs and an agent to be determined,
(iii) incubating the mixture obtained in (ii) for a predetermined time,
(iv) measuring the percentage of motile larvae having at least one PBMC or PMN attached, optionally at predetermined concentrations of the agent,
and optionally including at least one of the following:
(v) comparing the percentage obtained in (iv) with percentage of motile larvae having at least one PBMC or PMN attached in a control sample,
(vi) selecting the active agent which has an increased percentage over the control sample by at least 20%, and
(vii) determining an EC₅₀ value of the agent.

17. (canceled)

18. The in vitro assay according to claim 16, wherein a control sample is made in accordance with (i)-(iv) except that (ii) is performed in the absence of the agent.

19. (canceled)

20. The method according to claim 2, wherein the milbemycin is moxidectin.

21. The method according to claim 3, wherein the filariae or larvae thereof are selected from the group consisting of Dirofilaria immitis, Brugia malayi, Wuchereria bancrofti, Loa loa, Mansonella spp., Dirofilaria repens and Onchocerca volvulus.

22. The method according to claim 7, wherein the milbemycin is administered to the subject in need thereof in a dose adjusted to give a plasma concentration in the subject of 0.1-10 nM of milbemycin.

23. The method according to claim 22, wherein the milbemycin is administered to the subject in need thereof in a dose adjusted to give a plasma concentration in the subject of 0.5-3 nM of milbemycin.

24. The method according to claim 9, wherein the administration is subcutaneously, topically, or orally.

25. The method according to claim 11, wherein the milbemycin is moxidectin.

26. The method according to claim 13, wherein the milbemycin is moxidectin.
